(19) United States
(12) Patent Application Publication
(10) Pub. No.: US 2023/0145715 A1
(43) Pub. Date: May 11, 2023

(54) INSPECTION DEVICE FOR TOFU PRODUCTS, MANUFACTURING SYSTEM FOR TOFU PRODUCTS, INSPECTION METHOD FOR TOFU PRODUCTS, AND PROGRAM

(71) Applicant: TAKAI TOFU & SOYMILK EQUIPMENT CO., lshikawa (JP)

(72) Inventors: Toichiro TAKAI, lshikawa (JP); Motonari AMANO, lshikawa (JP); Yusuke SETO, lshikawa (JP)

(73) Assignee: TAKAI TOFU & SOYMILK EQUIPMENT CO., lshikawa (JP)

(21) Appl. No.: 17/906,942

(22) PCT Filed: Apr. 30, 2021
(86) PCT No.: PCT/JP2021/017304

§ 371 Date: Sep. 21, 2022

(30) Foreign Application Priority Data
Apr. 30, 2020 (JP) 2020-080296
Nov. 18, 2020 (JP) 2020-191601

(51) Int. Cl. G06T 7/00

(52) U.S. Cl. CPC G06T 7/0004; G06T 2207/20081; G06T 2207/30128

(57) ABSTRACT

An inspection device for tofu products includes a processor and a memory storing instructions that, when executed by the processor, cause a computer to execute operations. The operations include: acquiring a captured image from an image capturing device configured to capture an image of a tofu product to be inspected; and determining a quality of the tofu product indicated by the captured image using an evaluation value as output data obtained by inputting the captured image of the tofu product captured by the image capturing device as input data with respect to a learned model for determining a quality of a tofu product indicated by input data, the learned model being generated by performing machine learning using learning data including a captured image of a tofu product.

### TECHNICAL FIELD

The present invention relates to an inspection device for tofu products, a manufacturing system for tofu products, an inspection method for tofu products, and a program.

### BACKGROUND ART

According to related art, an inspection operation of detecting a non-defective product or a defective product among products in a production line and removing a product determined as the defective product from shipping objects has been performed as quality control on the products. Even at present when automation of production lines for products progresses, such an inspection operation often relies on human experience and visual observation, resulting in heavy human burden.

Regarding automation of a production line of such a product, various methods for improving a quality of the product have been disclosed. Patent Literature 1 discloses a device that inspects a shape defect by using a light cutting method for a rectangular parallelepiped product such as tofu or konjac. Patent Literature 2 discloses a technique of applying a method of deep learning and multivariate analysis by artificial intelligence (AI) in order to automatically sort a non-defective product or a defective product of food. Patent Literature 3 discloses that, in a machine for production such as frying, control parameters during production are learned by a neurosimulator as learning data, and information obtained as the learning result is used to determine control parameters during subsequent production. Patent Literature 4 discloses that, in detection of a foreign matter in food, a difference from an actual image during conveyance is calculated by using an identification unit that has been subjected to deep learning in advance such that image normalization data of only a non-defective product can be convoluted and a kernel image can be extracted from a neural network, and the foreign matter or the non-defective product is identified.

### CITATION LIST

### Patent Literature

Patent Literature 1: JP-A-2001-133233

Patent Literature 2: JP-A-2019-211288

Patent Literature 3: JP-A-H06-110863

Patent Literature 4: JP-A-2019-174481

### SUMMARY OF INVENTION

### Technical Problem

For example, it is assumed that a subtle change occurs in tofu, fried tofu, or the like depending on a production situation, a quality of a raw material, or the like. In addition, it is necessary to timely vary a determination criterion for determining the non-defective product or the defective product according to production conditions such as the number of products required for production or a disposal rate. According to the related art, such a determination is made by humans, and the determination criterion is also adjusted according to human experience or the like. Therefore, work by humans is required, and a workload is heavy. In the prior art described above, the tofu product cannot be inspected from a viewpoint of characteristics of the tofu product during production, and a load of manual inspection cannot be reduced.

In view of the above problems, an object of the present invention is to reduce a load of manual inspection while considering characteristics of tofu product during production.

### Solution to Problem

In order to solve the above problems, the present invention has the following configuration. That is, an inspection device for tofu products includes: an image capturing unit configured to capture an image of a tofu product to be inspected; and inspection means for determining a quality of the tofu product indicated by a captured image using an evaluation value as output data obtained by inputting the captured image of the tofu product captured by the image capturing unit as input data with respect to a learned model for determining a quality of a tofu product indicated by input data, the learned model being generated by performing machine learning using learning data including a captured image of a tofu product.

In addition, as another aspect of the present invention, the following configuration is provided. That is, an inspection method for tofu products includes: an acquisition step of acquiring a captured image of a tofu product to be inspected; and an inspection step of determining a quality of the tofu product indicated by the captured image using an evaluation value as output data obtained by inputting the captured image of the tofu product acquired in the acquisition step as input data with respect to a learned model for determining a quality of a tofu product indicated by input data, the learned model being generated by performing machine learning using learning data including the captured image of a tofu product.

In addition, as another aspect of the present invention, the following configuration is provided. That is, a program causes a computer to execute: an acquisition step of acquiring a captured image of a tofu product to be inspected; and an inspection step of determining a quality of the tofu product indicated by the captured image using an evaluation value as output data obtained by inputting the captured image of the tofu product acquired in the acquisition step as input data with respect to a learned model for determining a quality of a tofu product indicated by input data, the learned model being generated by performing machine learning using learning data including a captured image of a tofu product.

### Advantageous Effects of Invention

According to the present invention, it is possible to reduce a load of manual inspection while considering characteristics of a tofu product during production.

### BRIEF DESCRIPTION OF DRAWINGS

FIG. 1 is a schematic configuration view showing an example of an overall configuration of a manufacturing system for tofu products according to the present invention.

FIG. 2 is a schematic view showing conveyance of tofu products according to the present embodiment.

FIG. 3 is a block diagram showing an example of a functional configuration of a control device according to a first embodiment.

FIG. 4 is a conceptual diagram showing an overview of learning processing according to the first embodiment.

FIG. 5 is a flowchart of processing by the control device according to the first embodiment.

FIG. 6 is a conceptual diagram showing an overview of learning processing according to a second embodiment.

### DESCRIPTION OF EMBODIMENTS

Hereinafter, embodiments of the present invention will be described with reference to the drawings. The embodiments described below are embodiments for explaining the present invention, and are not intended to be interpreted as limiting the present invention. Moreover, not all configurations described in each embodiment are essential configurations for solving the problems of the present invention. In the drawings, the same components are denoted by the same reference numerals to indicate correspondence.

### First Embodiment

Hereinafter, a first embodiment of the present invention will be described.

First, characteristics of a tofu product as a product to be inspected according to the present invention during production will be described. The tofu product has characteristics that a shape and an appearance of the product easily varies due to influence of raw materials, a production environment, and the like. For example, an appearance of fried tofu, which is a kind of tofu products, may vary depending on a degree of expansion of an intermediate product, a degree of progress of deterioration of frying oil, or the like. Since the tofu product is also affected by the production environment, the shape and appearance of the product may vary depending on a production place, a daily environmental change, a state of a production machine, and the like. That is, the tofu product may have various shapes and appearances as compared with industrial products such as electronic devices.

When the product of the tofu product is manually inspected, a quality determination criterion is finely adjusted based on experience or the like in consideration of production conditions (the number of products required for production, a disposal rate, and the like) on the day. That is, the criterion for determining a quality of the tofu product may need to vary depending on a manufacturer, a production timing, and the like. Further, the tofu product may be manufactured in consideration of regional characteristics, a taste of the manufacturer or a purchaser, and the like, and the quality determination criterion may be diverse from such a viewpoint.

An inspection method for tofu products in consideration of the characteristics of a tofu product during production as described above will be described in a first embodiment of the present invention.

### Configuration Overview

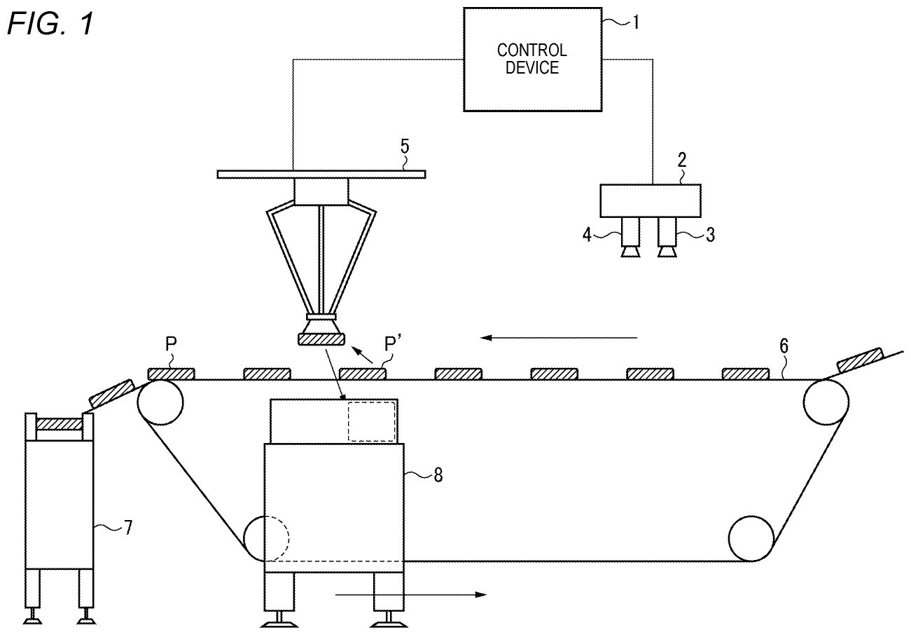

FIG. 1 is a schematic configuration view showing an overall configuration of a manufacturing system for tofu products (hereinafter, simply referred to as a “manufacturing system”) according to the present embodiment. The manufacturing system includes a control device 1, an inspection device 2, a removing device 5, a first conveyance device 6, a second conveyance device 7, and a storage device 8. Here, a product is collectively described as “a tofu product”, but more detailed classification included therein is not particularly limited. Examples of the tofu product may include deep-fried tofu, a deep-fried tofu pouch, thin deep-fried tofu, thick deep-fried tofu, a tofu cutlet, and a deep-fried tofu burger. Examples of the tofu may further include packaged silken tofu, silken tofu, cotton tofu, grilled tofu, and dried-frozen tofu. In addition, examples of the tofu product may include an intermediate product of the above tofu product, a product before or after packaging, or a product before or after cooling, freezing, or heating. In the following description, for the product (the tofu product), a product determined to have a certain quality or higher (that is, a non-defective product) is indicated by P, and a product determined to have a quality lower than the certain quality (that is, a defective product) is indicated by P′. When the product is comprehensively described, the reference numerals will be omitted.

The control device 1 controls an operation of the removing device 5 based on an image acquired by the inspection device 2. The inspection device 2 includes an image capturing unit 3 and an irradiation unit 4. The image capturing unit 3 includes an area camera such as a charge coupled device (CCD) camera or a complementary metal-oxide-semiconductor (CMOS) camera, or a line scan camera, and captures an image of a product being conveyed by the first conveyance device 6. The irradiation unit 4 irradiates the first conveyance device 6 (that is, a product to be inspected) with light in order to acquire a more appropriate image at the time of capturing an image by the image capturing unit 3. An image capturing operation of the inspection device 2 may be performed based on an instruction from the control device 1. Based on an instruction from the control device 1, the removing device 5 takes out the product P′ specified as a defective product from products being conveyed by the first conveyance device 6, and conveys the product P′ to the storage device 8.

FIG. 1 shows an example of a parallel link robot as the removing device 5, but a serial link robot may be used. A linear motion cylinder may be used as the removing device 5. The removing device 5 may include a hand-shaped gripping means including a plurality of finger portions, a holding means such as a vacuum suction pad type or a swirling airflow suction type, or the like. The removing device 5 may include a dual-arm robot, a collaborative robot, or the like. Since the removing device 5, the inspection device 2, and the like according to the present embodiment handle food such as tofu products, it is desirable that each has a certain quality according to, for example, an ingress protection standard (IP standard), which is a waterproof/dustproof standard for electronic devices. Specifically, a waterproof/dustproof grade having an IP standard of 54 or higher is preferable, and an IP of 65 or higher is more preferable.

The first conveyance device 6 conveys a plurality of products in a predetermined conveyance direction. The products to be conveyed here may be conveyed in one row or may be conveyed while being arranged in a plurality of rows. It is preferable that the products are arranged in a matrix or in a staggered manner, but the products may be randomly conveyed in a non-overlapping state. An inspection region of the inspection device 2 (that is, an image capturing region of the image capturing unit 3) is provided on a conveyance path of the first conveyance device 6.

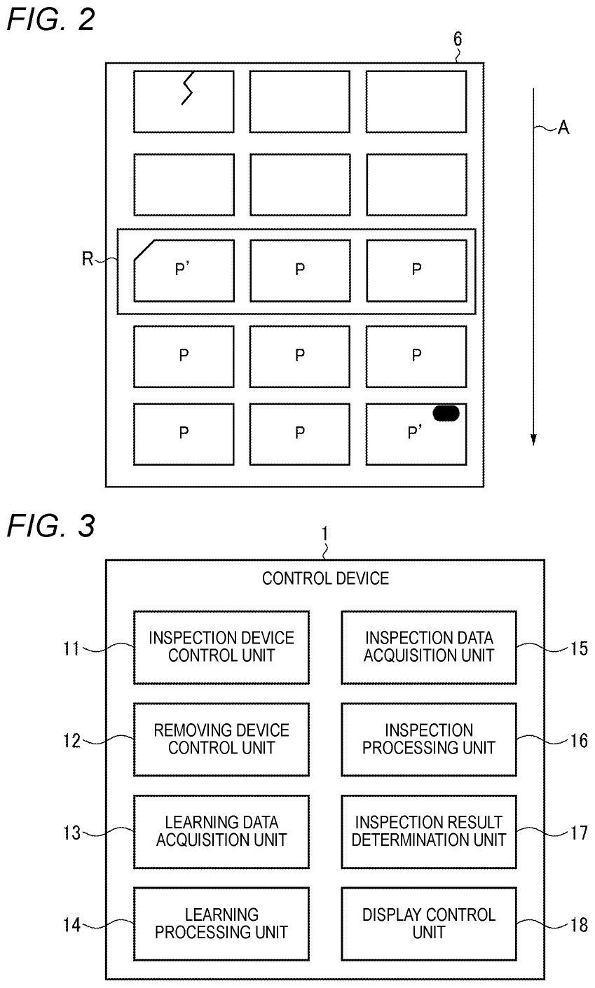

FIG. 2 is a conceptual view showing a state in which the products are being conveyed on the first conveyance device 6 according to the present embodiment. An arrow A shown in FIG. 2 indicates a conveyance direction of the products. A region R indicates an image capturing range of the image capturing unit 3, and is also a region with which light is irradiated by the irradiation unit 4. Here shows an example in which the products are being conveyed in three rows. A result of inspection on the products shows the product P determined as a non-defective product and the product P′ determined as a defective product. Examples of the defective product here include a product whose shape is chipped or cracked, a product in which foreign matter is detected on a surface, and the like.

The removing device 5 is configured to be operable in any of three axial directions (X axis, Y axis, and Z axis) such that the product P′ can be taken out on the conveyance path of the first conveyance device 6. Setting of an axial direction and an origin is not limited, and is omitted in the drawings. The first conveyance device 6 according to the present embodiment is formed of an endless belt, and the products are conveyed in the predetermined conveyance direction (for example, a direction of the arrow A in FIG. 2) by continuously rotating the endless belt. Although not shown in FIG. 1, it is assumed that a device that manufactures products is installed upstream of the first conveyance device 6 in the conveyance direction, and the manufactured products are sequentially conveyed. A state of the products conveyed by the first conveyance device 6 is not particularly limited, and may be, for example, a state of only the products before packaging or a state in which the products are packaged. That is, inspection according to the present embodiment may be performed on the products before packaging or on the products after packaging. Alternatively, inspection may be performed both before and after packaging.

The second conveyance device 7 receives the plurality of products P conveyed from the first conveyance device 6 and conveys the products P in a predetermined conveyance direction. In the example of FIG. 1, the conveyance direction of the first conveyance device 6 and the conveyance direction of the second conveyance device 7 are orthogonal to each other, and a matrix array is changed to a single row array for conveyance. A conveyance speed of the first conveyance device 6 and a conveyance speed of the second conveyance device 7 may be the same or different. Each of the first conveyance device 6 and the second conveyance device 7 may be configured as a conveyor type (for example, a belt conveyor, a net conveyor, a bar conveyor, a slat band chain, or the like), and is not particularly limited. Although not shown, the second conveyance device 7 may convey the products P (only non-defective products) in a stacked manner, may convey the products P in an inverted manner, or may convey the products P in an aligned manner. Thereafter, a conveyance device may be further provided, and an inspection device or a removing device may be further provided at an appropriate position. The conveyance device, the inspection device, or the removing device further provided in this case may have a configuration the same as that of the first conveyance device 6, the second conveyance device 7, the inspection device 2, or the removing device 5 described above, or may have a different configuration.

The storage device 8 stores the product P′ determined as the defective product. The stored product P′ may be conveyed to a different place via the storage device 8, or may be removed manually. The product P′ determined as the defective product may be discarded, or may be used for another purpose (for example, reproduction of an intermediate product, or a processed product such as chopped fried tofu).

The example of FIG. 1 shows a configuration in which the product P′ determined as the defective product is removed by the removing device 5, but the present invention is not limited thereto. For example, according to a ratio of the product P determined as the non-defective product and the product P′ determined as the defective product, the product P determined as the non-defective product may be taken out from the conveyed products by an alignment device (not shown) and conveyed to a subsequent conveyance device to be aligned. At this time, the alignment device (not shown) may perform operations such as packing the products P in a box and aligning a predetermined number of products P (for example, ten products in a case of fried tofu) in a vertical direction or a horizontal direction such that the products P are stacked. Alternatively, the product P′ determined as the defective product may be removed using the removing device 5, and the product P determined as the non-defective product may be conveyed from the first conveyance device 6 to the second conveyance device 7 using a relay device (not shown). Alternatively, a conveyance device that conveys products at regular intervals may be configured such that a branch is provided on a conveyance path, and conveyance is switched such that the product P determined as the non-defective product and the product P′ determined as the defective product proceed to different paths for sorting. A sorting function of removing or sorting the products according to such a determination result may be achieved by providing a mechanism such as a flipper type, an up-out type, a drop-out type, an air jet type, a trip type, a carrier type, a pusher type, a chute type, a shuttle type, a channelizer type, or a touchline selector type on the conveyance path.

The example of FIG. 1 shows a configuration in which the products are conveyed, sorted, and the like by devices in the manufacturing system, but the present invention is not limited thereto. For example, manual work may be performed as a part of sorting. For example, a worker may be notified by the manufacturing system such that the worker can visually confirm the product P′ determined as the defective product, and the worker works to remove the product P′. Notification here may be performed, for example, by displaying an image of the product P′ determined as the defective product on a display device (not shown), or may be performed by illuminating the product P′ with light or the like on the conveyance device. At this time, the worker may confirm the product notified by the manufacturing system and then determine whether to actually remove the product.

### Device Configuration

FIG. 3 is a block diagram showing an example of a functional configuration of the control device 1 according to the present embodiment. The control device 1 may be, for example, an information processing device such as a personal computer (PC). Each function shown in FIG. 3 may be achieved by a control unit (not shown) reading and executing a program for the function according to the present embodiment stored in a storage unit (not shown). The storage unit may include a random access memory (RAM), which is a volatile storage area, a read only memory (ROM), a hard disk drive (HDD), and the like, which are non-volatile storage areas. A central processing unit (CPU), a graphical processing unit (GPU), general-purpose computing on graphics processing units (GPGPU), or the like may be used as the control unit.

The control device 1 includes an inspection device control unit 11, a removing device control unit 12, a learning data acquisition unit 13, a learning processing unit 14, an inspection data acquisition unit 15, an inspection processing unit 16, an inspection result determination unit 17, and a display control unit 18.

The inspection device control unit 11 controls the inspection device 2 to control an image capturing timing and image capturing setting of the image capturing unit 3 and an irradiation timing and irradiation setting of the irradiation unit 4. The removing device control unit 12 controls the removing device 5 to remove the product P′ on the conveyance path of the first conveyance device 6 based on a determination result of whether the product is a non-defective product or a defective product.

The learning data acquisition unit 13 acquires learning data used in learning processing executed by the learning processing unit 14. Details of the learning data will be described later, and the learning data may be input based on, for example, an operation of an administrator of the manufacturing system. The learning processing unit 14 executes the learning processing using the acquired learning data to generate a learned model. Details of the learning processing according to the present embodiment will be described later. The inspection data acquisition unit 15 acquires an image captured by the inspection device 2 as inspection data. The inspection processing unit 16 applies the learned model generated by the learning processing unit 14 to the inspection data acquired by the inspection data acquisition unit 15 to inspect a product whose image is captured as the inspection data.

The inspection result determination unit 17 determines a control content for the removing device control unit 12 based on an inspection result of the inspection processing unit 16. Then, the inspection result determination unit 17 outputs a signal based on the determined control content to the removing device control unit 12. The display control unit 18 controls a display screen (not shown) displayed on a display unit (not shown) based on a determination result of the inspection result determination unit 17. The display screen (not shown) may display, for example, a statistical value of a product determined as a defective product based on the determination result of the inspection result determination unit 17, an actual image of the product P′ determined as the defective product, and the like.

### Learning Processing

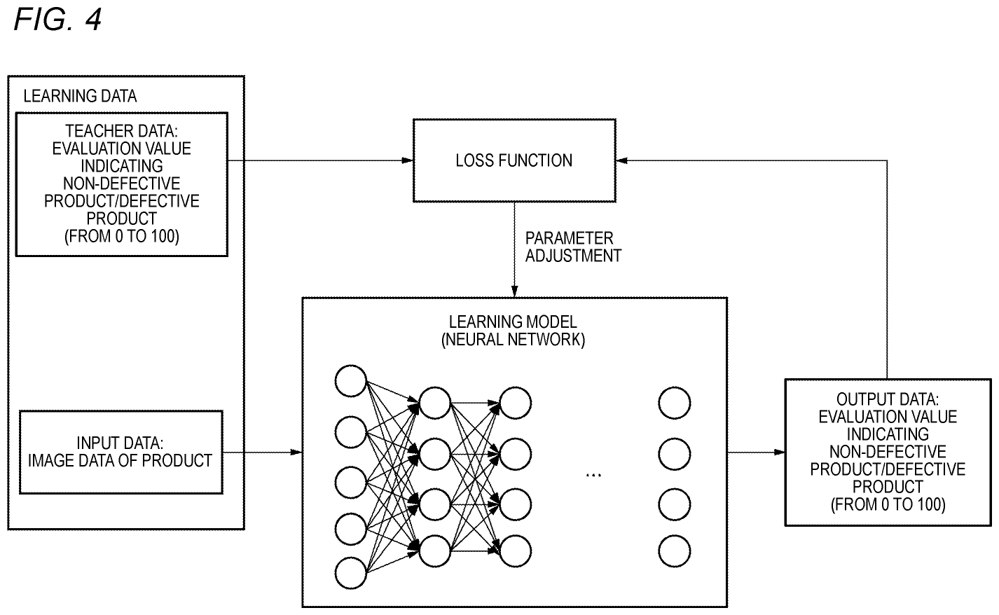

In the present embodiment, a method of deep learning using a neural network among machine learning is used as a learning method, and supervised learning will be described as an example. Amore specific method (algorithm) of deep learning is not particularly limited, and for example, a known method such as a convolutional neural network (CNN) may be used. FIG. 4 is a schematic diagram showing a concept of the learning processing according to the present embodiment. The learning data used in the present embodiment includes a pair of image data of a product as input data and an evaluation value obtained by evaluation of humans (a manufacturer of tofu products) on the product as teacher data. Here, values from 0 to 100 are set as evaluation values, and the larger the number is, the higher the evaluation is. A granularity of the evaluation values is not limited thereto, and for example, the evaluation may be performed in three stages A, B, and C, or by two values of non-defective product/defective product, or may be performed by an evaluation value for each of a plurality of defective product items. A method for normalizing the evaluation value for the product is not limited to the above, and other classifications may be used. The machine learning other than the neural network is not particularly limited as long as it is machine learning in a broad sense such as a decision tree, a support vector machine, a random forest, and a regression analysis (multivariate analysis, multiple regression analysis).

When input data prepared as learning data (here, image data of a tofu product) is input to a learning model, an evaluation value is output as output data for the input data. Next, an error is derived by a loss function using the output data and teacher data prepared as learning data (here, the evaluation value for the tofu product indicated by the image data). Then, parameters in the learning model are adjusted so as to reduce the error. For example, an error back propagation method or the like may be used to adjust the parameters. In this way, a learned model is generated by repeatedly performing learning using a plurality of learning data.

The learning model used in the present embodiment may have a configuration in which learning is performed using learning data from a state in which learning is not performed at all. However, in order to obtain an optimum learned model, a large amount of learning data is required, and a processing load due to repetition of learning processing using the learning data is also heavy. Therefore, a user (for example, the manufacturer of tofu products) may be burdened by updating the learned model with new learning data. Therefore, for a purpose of identifying an image, parameters of a learning model in which a certain degree of learning has progressed may be used for a huge number of image data. A learning model in which learning processing by deep learning has progressed in view of image recognition includes a part that can be commonly used even when a target of image recognition is different. In a learning model reinforced by the image recognition, adjustment of parameters in convolution layers and pooling layers including several tens to several hundreds of layers has already progressed. In the present embodiment, for example, a so-called transfer learned learning model may be used in which values of parameters of most of convolutional layers from an input side are fixed without being changed, and new learning data (for example, an image of a tofu product) is learned for several layers (for example, only the last one to several layers) on an output side to adjust parameters. When such a transfer learning model is used, the number of new learning data is relatively small, and there is an advantage that it is possible to easily update the learned model while reducing a processing load of relearning.

The learning processing does not necessarily have to be executed by the control device 1. For example, the manufacturing system may be configured to provide learning data to a learning server (not shown) provided outside the manufacturing system and execute learning processing on a server side. Then, the server may provide a learned model to the control device 1 if necessary. Such a learning server may be located on a network (not shown) such as the Internet, and the server and the control device 1 are communicably connected to each other.

### Processing Flow

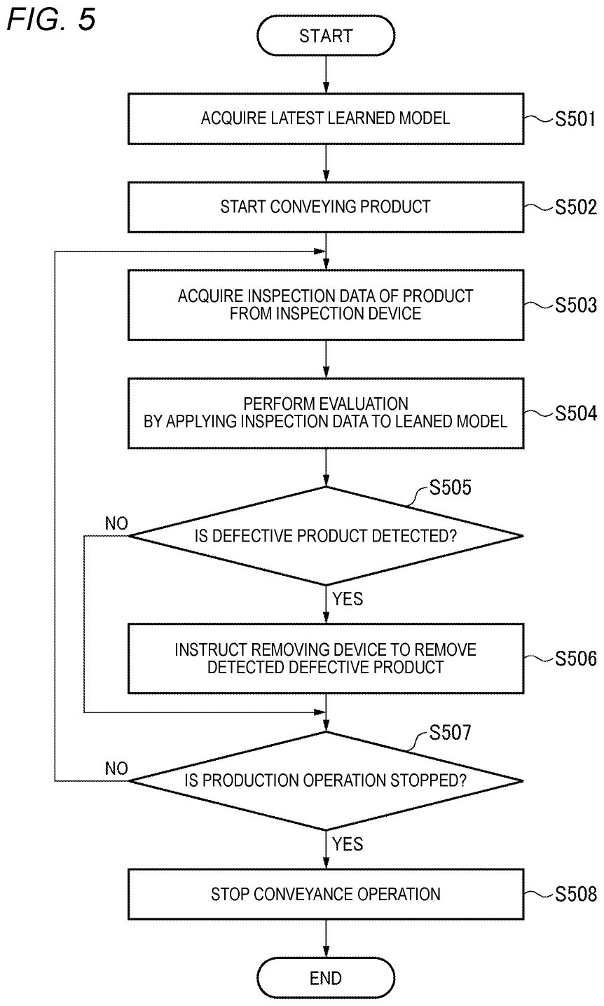

Hereinafter, a processing flow of the control device 1 according to the present embodiment will be described with reference to FIG. 5. Processing to be described below is implemented, for example, by a CPU (not shown) or a GPU (not shown) included in the control device 1 reading and executing a program stored in a storage device (not shown) such as an HDD. The following processing may be continuously executed while the manufacturing system is operating.

In S501, the control device 1 acquires the latest learned model among learned models generated by executing learning processing. The learned model is updated each time the learning processing is timely repeated for a learning model. Therefore, the control device 1 acquires the latest learned model when the present processing is started, and uses the latest learned model in the subsequent processing.

In S502, the control device 1 causes the inspection device 2 to start capturing an image on a conveyance path of the first conveyance device 6. Further, the control device 1 operates the first conveyance device 6 and the second conveyance device 7 to start conveying a product.

In S503, the control device 1 acquires inspection data (an image of the product) transmitted timely from the inspection device 2 in accordance with conveyance of the product by the first conveyance device 6. When a conveyance interval between conveyed products or a conveyance position where each product is arranged is defined in advance on the conveyance path, the image of the product may be separately captured based on the position. Alternatively, when the inspection data transmitted timely from the inspection device 2 is a moving image, frames may be extracted from the moving image at predetermined intervals, and the frame may be treated as image data. Captured raw image data may be used directly as the image of the product. The raw image data may be used as learning data by being appropriately subjected to data cleansing processing (excluding data whose characteristics are difficult for humans to view) or padding processing (a plurality of images with increased noise or a plurality of images with adjusted brightness are also added to the learning data). Processed image data obtained by applying certain image processing to the raw image data may be used as the learning data. The certain image processing may include, for example, various types of filter processing such as contour processing (edge processing), position correction processing (rotation, center position movement, and the like), brightness correction, shading correction, contrast conversion, convolution processing, difference (primary differential, secondary differential), binarization, noise removal (smoothing), contour smoothing, real-time shading correction, blurring processing, real-time difference, contrast expansion, filter coefficient processing (averaging, median, shrinkage, expansion), and the like. The preprocessing and data processing have advantages such as reduction and adjustment of the number of learning data, improvement of learning efficiency, reduction of disturbance influence, and the like.

In S504, the control device 1 inputs the inspection data (the image data of the product) acquired in S503 to the learned model. Thereby, an evaluation value of the product indicated by the inspection data is output as output data. It is determined whether the product to be inspected is a non-defective product or a defective product according to the evaluation value.

In S505, the control device 1 determines whether the product to be inspected is a defective product based on the evaluation value obtained in S504. When the defective product is detected (YES in S505), the processing of the control device 1 proceeds to S506. On the other hand, when the defective product is not detected (NO in S505), the processing of the control device 1 proceeds to S507.

For example, in a configuration in which the evaluation value is evaluated by 0 to 100, a threshold value for the evaluation value may be set, and it may be determined whether the product to be inspected is the non-defective product or the defective product by comparing the threshold value with the evaluation value output from the learned model. In this case, the threshold value serving as a criterion for determining whether the product is the non-defective product or the defective product may be set by an administrator of the manufacturing system (for example, a manufacturer of tofu products) via a setting screen (not shown) at any timing. As described above, an appearance and a shape of the tofu product to be inspected in the present embodiment may change depending on various factors. In consideration of such a change, the administrator may be able to control the threshold value for the output data obtained by the learned model. In a configuration in which the evaluation value is evaluated by A, B, and C, the evaluation values A and B may be treated as non-defective products, and the evaluation value C may be treated as a defective product. At this time, the product having the evaluation value A may be treated as a non-defective product, and the product having the evaluation value B may be treated as a quasi-non-defective product. A plurality of threshold values may be set, and the threshold values may be used to determine quasi-non-defective products graded between a non-defective product and a defective product.

In S506, the control device 1 controls the removing device 5 by instructing the removing device 5 to remove the product detected as the defective product in S505. At this time, in order to remove the product P′ detected as the defective product, the control device 1 specifies a position of the product P′ to be removed based on the inspection data acquired from the inspection device 2, a conveyance speed of the first conveyance device 6, and the like. As a method for specifying the position of the product, a known method may be used, and detailed description thereof will be omitted here. The removing device 5 conveys the product P′ to be removed to the storage device 8 based on an instruction from the control device 1.

Even when a quality of the appearance of the tofu product does not satisfy a certain criterion, the tofu product may be used as a raw material for another processed product. Therefore, for example, in a configuration in which the evaluation value is evaluated by A, B, and C, the evaluation value A may be treated as a non-defective product, the evaluation value B may be treated as a processing target, and the evaluation value C may be treated as a defective product. Alternatively, in a case of being diverted for processing, more classifications may be used according to the diverting destination. In this case, the control device 1 may control the removing device 5 such that the product determined to have the evaluation value B is stored in a storage device (not shown) for a processed product. Examples of the processed product to be diverted include manufacturing chopped fried tofu from fried tofu, manufacturing ganmodoki from tofu, and mixing finely pasted liquid (reproduced liquid) with a soybean juice or soymilk for reuse.

In S507, the control device 1 determines whether a production operation is stopped. Stop of the production operation may be determined in response to detection that supply of the product from an upstream side of the first conveyance device 6 is stopped, or may be determined based on a notification from the upstream device. When the production operation is stopped (YES in S507), the processing of the control device 1 proceeds to S508. On the other hand, when the production operation is not stopped (NO in S507), the processing of the control device 1 returns to S503, and the corresponding processing is repeated.

In S508, the control device 1 stops a conveyance operation of the first conveyance device 6. The control device 1 may perform an operation of executing initialization processing on the learned model acquired in S501. Then, the present processing flow is ended.

The inspection data acquired in S503 may be stored for use in future learning processing. In this case, image processing may be executed such that the acquired inspection data becomes image data for learning.

### Display Processing

In the present embodiment, when an image of the product P′ determined as the defective product is displayed on the display unit (not shown) as a result of inspection on the product of the tofu product, a basis (defective portion) for determination as the defective product may be displayed. In learning of the neural network as described above, there is a visualization method such as GRAD-CAM or Guided Grad-CAM. By using such a method, when a product to be inspected is determined as a defective product, a focused region may be specified as a basis for determination, and the region may be visualized and displayed. Even in a case of a product determined as a non-defective product, when an evaluation value therefor is close to an evaluation value for determination as the defective product, a focused region may be specified and displayed using the above-described method.

As described above, according to the present embodiment, it is possible to reduce a load of manual inspection while considering the characteristics of tofu product during production.

For the tofu product whose appearance is easily affected by the production environment, the raw materials, and the like, the manufacturer (for example, the administrator of the manufacturing system) can reflect a criterion for determining whether a product is a non-defective product or a defective product depending on a situation, and thus the quality can be determined depending on the manufacturer.

### Second Embodiment

Hereinafter, a second embodiment of the present invention will be described. An example in which supervised learning is used as learning processing has been described in the first embodiment. In contrast, an example in which unsupervised learning is used as learning processing will be described in the second embodiment of the present invention. Description of a configuration the same as that of the first embodiment will be omitted, and description will be made focusing on a difference.

### Learning Processing

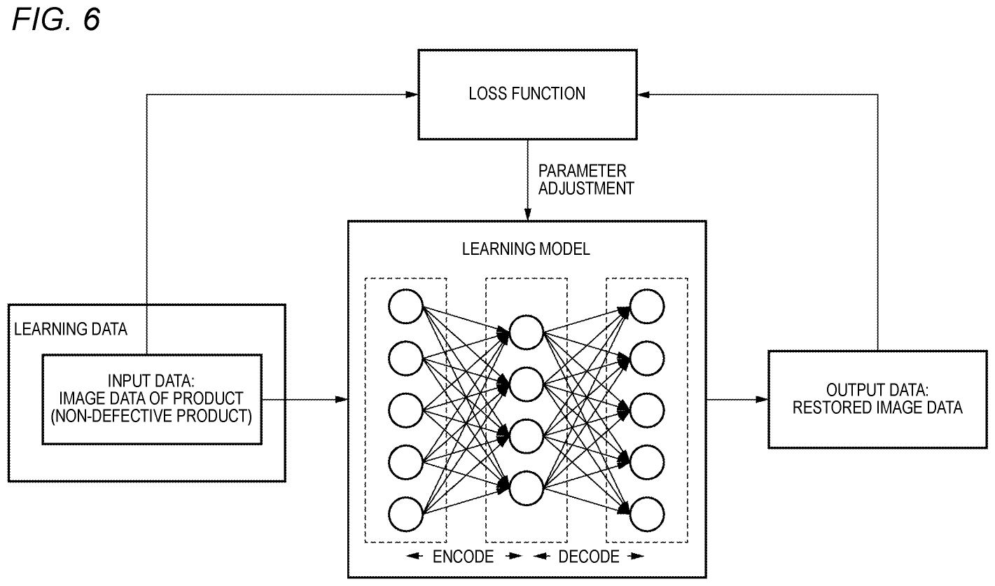

In the present embodiment, a method of deep learning using a neural network among machine learning is used as a learning method, and unsupervised learning will be described as an example. A more specific method (algorithm) of deep learning is not particularly limited, and a known method such as a variational auto-encoder (VAE) may be used. FIG. 6 is a schematic diagram showing a concept of the learning processing according to the present embodiment.

Learning data used in the present embodiment is image data of a product. Only image data of a product (a tofu product) determined as a non-defective product by the administrator of the manufacturing system (for example, the manufacturer of tofu products) is used as the image data here. In related art, it is difficult to prepare all teacher data (image data) of variations indicating products to be determined as defective products. Therefore, in the present embodiment, learning is performed using only the image data of the non-defective product, and a learned model for determining whether a product is a non-defective product is generated.

A learning model according to the present embodiment includes an encoder and a decoder. The encoder generates vector data having a plurality of dimensions by using input data. The decoder restores the image data using the vector data generated by the encoder.

When the input data prepared as the learning data (here, the image data of a tofu product (non-defective product)) is input to the learning model, the restored image data of the tofu product (non-defective product) is output as output data for the input data by operations of the encoder and the decoder. Next, the output data and the original input data (that is, the image data of the tofu product (non-defective product)) are used to derive an error by a loss function. Then, parameters of the encoder and the decoder in the learning model are adjusted so as to reduce the error. For example, an error back propagation method or the like may be used to adjust the parameters. By repeatedly performing learning using a plurality of learning data in this manner, the learned model capable of restoring the image data of the tofu product (non-defective products) is generated.

In the present embodiment, a detection function of detecting a defective product using the learned model is achieved. Image data of the tofu product is input to the learned model, restored image data obtained as an output of the image data is compared with the input image data, and when a difference between the restored image data and the input image data is larger than a predetermined threshold value, the tofu product indicated by the input image data is determined as a defective product. On the other hand, when the difference is equal to or smaller than the predetermined threshold value, the tofu product indicated by the input image data is determined as a non-defective product. In other words, it is determined whether a product indicated by the input image data is the defective product based on how much difference exists from image data of tofu product determined as a non-defective product. The threshold value here may be a threshold value for a size (for example, the number of pixels) of a region to be a difference, or may be a threshold value for the number of regions to be a difference. Alternatively, a difference in pixel values (RGB values) on an image may be used.

The number of dimensions of the vector data (latent variable) in an intermediate stage of the learning model is not particularly limited, and may be specified by the administrator of the manufacturing system (for example, the manufacturer of tofu products) or may be determined using a known method. The number of dimensions may be determined according to a processing load or detection accuracy.

### Processing Flow

A processing flow according to the present embodiment is basically the same as the processing flow described with reference to FIG. 5 in the first embodiment. At this time, it is assumed that the learning processing by the unsupervised learning as shown in FIG. 6 has already been performed, and the learned model has been generated. A difference between the processing is a content of the processing in S504.

In S504, the control device 1 inputs the image data indicating the product to be inspected to the learned model generated by the unsupervised learning. As a result, restored image data is obtained. The control device 1 obtains a difference between the reproduced image data and the input image data. When the difference is larger than a predetermined threshold, the control device 1 determines that the tofu product indicated by the input image data is a defective product. On the other hand, when the difference is equal to or smaller than the predetermined threshold value, the control device 1 determines that the tofu product indicated by the input image data is a non-defective product. The difference may be calculated using a loss function shown in FIG. 6. That is, the difference can be handled as an evaluation value for the input image data. The predetermined threshold used for determination may be set to any value at any timing by the administrator of the manufacturing system (for example, the manufacturer of tofu products), or may be set based on a predetermined condition by the manufacturing system. The setting condition here may be set based on, for example, the number of products required to be manufactured, a discard rate, or the like.

### Display Processing

In the present embodiment, when an image of the product P′ determined as a defective product, a quasi-non-defective product, or the like instead of a non-defective product is displayed on the display unit (not shown) as a result of inspection on a product of a tofu product, a basis or a cause for determination as the defective product or the quasi-non-defective product may be displayed. In the auto-encoder as described above, a position corresponding to the difference between the input data and the output data can be specified by comparing the input data with the output data. An icon (such as a red circle) may be added to the specified position or the specified position may be color-coded to be visualized and displayed.

In the present embodiment, learning is performed using only the image data of the tofu product (non-defective product), and a product of a tofu product is determined as a non-defective product or a defective product using the learned model obtained as a result of the learning.

In the present embodiment, the image data indicating the product P determined as the non-defective product in the step S504 may be stored so as to be used as subsequent learning data. In this case, whether the stored image data is used as the learning data may be presented to the administrator of the manufacturing system in a selectable manner.

As described above, according to the present embodiment, by using the unsupervised learning, it is possible to reduce time and effort related to generation of learning data in addition to effects of the first embodiment.

### Other Embodiments

In the above embodiment, the inspection device 2 is configured to capture an image of only one surface (upper surface in FIG. 1) of the product for inspection as shown in FIG. 1. However, the present invention is not limited thereto, and, for example, an image of a back surface or a side surface may be acquired and inspected in addition to a front surface. In this case, a plurality of inspection devices 2 may be provided, and images of a product may be captured from a plurality of directions by image capturing units (cameras) included in the plurality of respective inspection devices 2. For example, a first image capturing unit (not shown) may be installed so as to capture an image of a front surface of the product from a first direction, and a second image capturing unit (not shown) may be installed so as to capture an image of a back surface of the product from a second direction. Alternatively, a configuration (inversion mechanism) that inverts a product on a conveyance path may be provided in the first conveyance device 6, and images of the product may be captured before and after inversion, and inspection may be performed using the captured images. At this time, the inspection may be performed using different learned models for a front surface, a back surface, and a side surface of the product. That is, learning is performed using different learning data of the front surface, the back surface, and the side surface according to a type, a packaging state, and the like of the product conveyed by the first conveyance device 6, and thus the learned models corresponding to respective surfaces are generated. Then, the inspection may be performed using the learned models corresponding to image capturing directions.

In the above embodiment, the irradiation unit 4 irradiates the product with light from a direction the same as that of the image capturing unit 3 (camera) as shown in FIG. 1. However, the present invention is not limited to this configuration, and for example, the image capturing unit 3 and the irradiation unit 4 may have different positions and orientations facing the product. In a case of this configuration, for example, the irradiation unit 4 may include a light source that irradiates the product with a wavelength of infrared rays, and the image capturing unit 3 may acquire image data based on transmitted light, transmitted reflected light, or transmitted scattered light of the product. Then, the product may be inspected based on internal information on the product indicated by the image data.

As described above, the following matters are disclosed in the present specification.

(1) An inspection device for tofu products, including:

an image capturing unit configured to capture an image of a tofu product to be inspected; and

inspection means for determining a quality of the tofu product indicated by a captured image using an evaluation value as output data obtained by inputting the captured image of the tofu product captured by the image capturing unit as input data with respect to a learned model for determining a quality of a tofu product indicated by input data, the learned model being generated by performing machine learning using learning data including a captured image of a tofu product.

According to this configuration, it is possible to reduce a load of manual inspection while considering characteristics of the tofu product during production.

(2) The inspection device for tofu products according to (1),

wherein the inspection means compares the evaluation value of the input data with a predetermined threshold value to determine the quality of the tofu product indicated by the input data by a plurality of classifications including a non-defective product.

According to this configuration, the quality of the tofu product can be determined by the plurality of classifications including the non-defective product based on the preset threshold value.

(3) The inspection device for tofu products according to (2), further including:

setting means for receiving setting of the predetermined threshold value.

According to this configuration, a manufacturer of the tofu product can set as desired the threshold value as a criterion used for determining whether the tofu product is a non-defective product or a defective product.

(4) The inspection device for tofu products according to any one of (1) to (3), further including:

learning processing means for newly generating and updating the learned model by repeatedly performing machine learning using a new (unknown, unlearned) captured image of a tofu product.

According to this configuration, the inspection device for tofu products can update the learned model for new captured image data having an unknown (unlearned) evaluation value, and can execute learning processing according to the tofu product to be inspected.

(5) The inspection device for tofu products according to any one of (1) to (4),

wherein the machine learning is supervised learning using learning data in which a captured image of a tofu product and an evaluation value corresponding to a quality of the tofu product indicated by the captured image are paired.

According to this configuration, inspection by the supervised learning can be performed using the learning data based on a set value set by the manufacturer of the tofu product.

(6) The inspection device for tofu products according to (5),

wherein the evaluation value is a value expressed by a score in a predetermined range.

According to this configuration, the manufacturer of the tofu product can normalize and set an evaluation value in any range for the tofu product and use the normalized set evaluation value as learning data, and can acquire an inspection result based on the learning data.

(7) The inspection device for tofu products according to any one of (1) to (3),

wherein the machine learning is unsupervised learning using a captured image indicating a non-defective product of a tofu product as learning data.

According to this configuration, the manufacturer of the tofu product may prepare only the image data of the tofu product that is the non-defective product, and a load for preparing data required for learning can be reduced.

(8) The inspection device for tofu products according to any one of (1) to (7), further including:

display means for displaying a captured image indicating a tofu product determined as a classification different from a non-defective product, based on an inspection result of the inspection means.

According to this configuration, the manufacturer of the tofu product can confirm an image of the actual tofu product determined as the classification different from the non-defective product.

(9) The inspection device for tofu products according to (8),

wherein the display means specifies and displays a portion of the captured image indicating the tofu product determined as a defective product, the portion causing a determination as the classification different from a non-defective product.

According to this configuration, the manufacturer of the tofu product can more clearly confirm the image of the actual tofu product determined as the classification different from the non-defective product and the cause therefor.

(10) The inspection device for tofu products according to any one of (1) to (9),

wherein the image capturing unit includes:
a first image capturing unit configured to capture an image of the tofu product from a first direction; and
a second image capturing unit configured to capture an image of the tofu product from a second direction different from the first direction, and

wherein the inspection means uses images captured by the first image capturing unit and the second image capturing unit as the input data.

According to this configuration, the tofu product can be inspected from a plurality of viewpoints, and the inspection can be performed with higher accuracy.

(11) The inspection device for tofu products according to (10),

wherein the first direction is a direction for capturing the image of a front surface of the tofu product, and

wherein the second direction is a direction for capturing the image of a back surface of the tofu product.

According to this configuration, by inspecting the front surface and the back surface of the tofu product, the inspection can be performed with higher accuracy.

(12) The inspection device for tofu products according to (10) or (11),

wherein, in the inspection means, a learned model in a case where a captured image captured by the first image capturing unit is used as the input data is different from a learned model in a case where a captured image captured by the second image capturing unit is used as input data.

According to this configuration, by switching the learned model to be used according to a direction in which the tofu product is to be inspected, the inspection can be performed according to the direction, and thus can be performed with higher accuracy.

(13) The inspection device for tofu products according to any one of (1) to (12),

wherein the tofu product is any one of packaged silken tofu, silken tofu, cotton tofu, grilled tofu, dried-frozen tofu, deep-fried tofu, a deep-fried tofu pouch, thin deep-fried tofu, thick deep-fried tofu, a tofu cutlet, and a deep-fried tofu burger.

According to this configuration, the tofu product can be inspected corresponding to a specific type of product.

(14) A manufacturing system for tofu products, including:

the inspection device for tofu products according to any one of (1) to (13);

a conveyance device configured to convey tofu products; and

a sorting mechanism configured to sort the tofu products conveyed by the conveyance device based on an inspection result of the inspection device for tofu products.

According to this configuration, it is possible to provide the manufacturing system for tofu products that reduces a load of manual inspection and sorting of products according to the quality while considering characteristics of the tofu product during production.

(15) The manufacturing system for tofu products according to (14), further including:

an alignment device configured to align the tofu products sorted by the sorting mechanism according to a predetermined rule based on the inspection result of the inspection device for tofu products.

According to this configuration, it is possible to provide the manufacturing system for tofu products that reduces a load of manual inspection and alignment of products according to the quality while considering the characteristics of the tofu product during production.

(16) An inspection method for tofu products, including:

an acquisition step of acquiring a captured image of a tofu product to be inspected; and

an inspection step of determining a quality of the tofu product indicated by the captured image using an evaluation value as output data obtained by inputting the captured image of the tofu product acquired in the acquisition step as input data with respect to a learned model for determining a quality of a tofu product indicated by input data, the learned model being generated by performing machine learning using learning data including a captured image of a tofu product.

According to this configuration, it is possible to reduce a load of manual inspection while considering characteristics of the tofu product during production.

(17) A program for causing a computer to execute:

an acquisition step of acquiring a captured image of a tofu product to be inspected; and

an inspection step of determining a quality of the tofu product indicated by the captured image using an evaluation value as output data obtained by inputting the captured image of the tofu product acquired in the acquisition step as input data with respect to a learned model for determining a quality of a tofu product indicated by input data, the learned model being generated by performing machine learning using learning data including a captured image of a tofu product.

According to this configuration, it is possible to reduce a load of manual inspection while considering characteristics of the tofu product during production.

Although various embodiments have been described above with reference to the drawings, it is needless to say that the present invention is not limited to these examples. It is apparent that those skilled in the art can conceive of various modifications and alterations within the scope of the claims, and it is understood that such modifications and alterations naturally fall within the technical scope of the present invention. Components in the embodiments described above may be combined within a range not departing from the spirit of the present invention.

The present application is based on a Japanese patent application filed on Apr. 30, 2020 (Japanese Patent Application No. 2020-080296) and a Japanese patent application filed on Nov. 18, 2020 (Japanese Patent Application No. 2020-191601), and the contents thereof are incorporated herein as reference.

### REFERENCE SIGNS LIST

1: Control Device

2: Inspection Device

3: Image Capturing Unit

4: Irradiation Unit

5: Removing Device

6: First Conveyance Device

7: Second Conveyance Device

8: Storage Device

P: Product (Non-Defective Product)

P′: Product (Defective Product)

11: Inspection Device Control Unit

12: Removing Device Control Unit

13: Leaning Data Acquisition Unit

14: Learning Processing Unit

15: Inspection Data Acquisition Unit

16: Inspection Processing Unit

17: Inspection Result Determination Unit

18: Display Control Unit

1. An inspection device for tofu products, comprising:
a processor; and
a memory storing instructions that, when executed by the processor, cause a computer to execute operations comprising:
acquiring a captured image from an image capturing device configured to capture an image of a tofu product to be inspected; and
determining a quality of the tofu product indicated by the captured image using an evaluation value as output data obtained by inputting the captured image of the tofu product captured by the image capturing device as input data with respect to a learned model for determining a quality of a tofu product indicated by input data, the learned model being generated by performing machine learning using learning data including a captured image of a tofu product.

2. The inspection device for tofu products according to claim 1,
wherein the determining the quality of the tofu product comprises comparing the evaluation value of the input data with a predetermined threshold value to determine the quality of the tofu product indicated by the input data by a plurality of classifications including a non-defective product.

3. The inspection device for tofu products according to claim 2,
wherein the operations further comprise receiving setting of the predetermined threshold value.

4. The inspection device for tofu products according to claim 1,
wherein the operations further comprise newly generating and updating the learned model by repeatedly performing machine learning using a new captured image of a tofu product.

5. The inspection device for tofu products according to claim 1,
wherein the machine learning is supervised learning using learning data in which a captured image of a tofu product and an evaluation value corresponding to a quality of the tofu product indicated by the captured image are paired.

6. The inspection device for tofu products according to claim 5,
wherein the evaluation value is a value expressed by a score in a predetermined range.

7. The inspection device for tofu products according to claim 1,
wherein the machine learning is unsupervised learning using a captured image indicating a non-defective product of a tofu product as learning data.

8. The inspection device for tofu products according to claim 1,
wherein the operations further comprise displaying a captured image indicating a tofu product determined as a classification different from a non-defective product, based on a determination result of the quality of the tofu product.

9. The inspection device for tofu products according to claim 8,
wherein the displaying the captured image comprises specifying and displaying a portion of the captured image indicating the tofu product determined as the classification different from the non-defective product, the portion causing a determination as the classification different from the non-defective product.

10. The inspection device for tofu products according to claim 1,
wherein the image capturing device comprises:
a first image capturing device configured to capture an image of the tofu product from a first direction; and
a second image capturing device configured to capture an image of the tofu product from a second direction different from the first direction, and
wherein the determining the quality of the tofu product comprises using images captured by the first image capturing device and the second image capturing device as the input data.

11. The inspection device for tofu products according to claim 10,
wherein the first direction is a direction for capturing the image of a front surface of the tofu product, and
wherein the second direction is a direction for capturing the image of a back surface of the tofu product.

12. The inspection device for tofu products according to claim 10,
wherein, in the determining the quality of the tofu product, a learned model in a case where a captured image captured by the first image capturing device is used as the input data is different from a learned model in a case where a captured image captured by the second image capturing device is used as input data.

13. The inspection device for tofu products according to claim 1,
wherein the tofu product is any one of packaged silken tofu, silken tofu, cotton tofu, grilled tofu, dried-frozen tofu, deep-fried tofu, a deep-fried tofu pouch, thin deep-fried tofu, thick deep-fried tofu, a tofu cutlet, and a deep-fried tofu burger.

14. A manufacturing system for tofu products comprising:
the inspection device for tofu products according to claim 1;
a conveyance device configured to convey tofu products; and
a sorting mechanism configured to sort the tofu products conveyed by the conveyance device based on an inspection result of the inspection device for tofu products.

15. The manufacturing system for tofu products according to claim 14, further comprising:
an alignment device configured to align the tofu products sorted by the sorting mechanism according to a predetermined rule based on the inspection result of the inspection device for tofu products.

16. An inspection method for tofu products, comprising:
acquiring a captured image of a tofu product to be inspected; and
determining a quality of the tofu product indicated by the captured image using an evaluation value as output data obtained by inputting the captured image as input data with respect to a learned model for determining a quality of a tofu product indicated by input data, the learned model being generated by performing machine learning using learning data including a captured image of a tofu product.

17. A non-transitory computer-readable medium storing a program that, when executed by a processor, causes a computer to execute operations comprising:
acquiring a captured image of a tofu product to be inspected; and
determining a quality of the tofu product indicated by the captured image using an evaluation value as output data obtained by inputting the captured image as input data with respect to a learned model for determining a quality of a tofu product indicated by input data, the learned model being generated by performing machine learning using learning data including a captured image of a tofu product.

18. The inspection device for tofu products according to claim 1,
wherein the operations further comprise:
displaying a captured image and a statistical value of determination, the captured image indicating a tofu product determined as a classification of a plurality of classifications, the classification being different from a non-defective product, based on a determination result of the quality of the tofu product; and
receiving setting of a plurality of values as predetermined threshold values, the plurality of values being in a range of the evaluation value obtained as the output data,
wherein the determining the quality of the tofu product comprises comparing the evaluation value of the input data with the predetermined threshold values to determine the quality of the tofu product indicated by the input data by a plurality of classifications including a non-defective product and a defective product, and
wherein the displaying the captured image and the statistical value of determination comprises specifying and displaying a portion of the captured image indicating the tofu product determined as the classification of the plurality of classifications different from the non-defective product, the portion causing a determination as the classification different from the non-defective product.

19. The inspection device for tofu products according to claim 1,
wherein the machine learning uses a learning model by transfer learning.

20. The inspection method for tofu products according to claim 16, further comprising:
displaying a captured image and a statistical value of determination, the captured image indicating a tofu product determined as a classification of a plurality of classifications, the classification being different from a non-defective product, based on a determination result of the quality of the tofu product; and
receiving setting of a plurality of values as predetermined threshold values, the plurality of values being in a range of the evaluation value obtained as the output data,
wherein the determining the quality of the tofu product comprises comparing the evaluation value of the input data with the predetermined threshold values to determine the quality of the tofu product indicated by the input data by a plurality of classifications including a non-defective product and a defective product, and
wherein the displaying the captured image and the statistical value of determination comprises specifying and displaying a portion of the captured image indicating the tofu product determined as the classification of the plurality of classifications different from the non-defective product, the portion causing a determination as the classification different from the non-defective product.

21. The non-transitory computer-readable medium according to claim 17,
wherein the operations further comprise:
displaying a captured image and a statistical value of determination, the captured image indicating a tofu product determined as a classification of a plurality of classifications, the classification being different from a non-defective product, based on a determination result of the quality of the tofu product; and
receiving setting of a plurality of values as predetermined threshold values, the plurality of values being in a range of the evaluation value obtained as the output data,
wherein the determining the quality of the tofu product comprises comparing the evaluation value of the input data with the predetermined threshold values to determine the quality of the tofu product indicated by the input data by a plurality of classifications including a non-defective product and a defective product, and
wherein the displaying the captured image and the statistical value of determination comprises specifying and displaying a portion of the captured image indicating the tofu product determined as the classification of the plurality of classifications different from the non-defective product, the portion causing a determination as the classification different from the non-defective product.
